(19) United States
(12) Patent Application Publication
(10) Pub. No.: US 2022/0243153 A1
(43) Pub. Date: Aug. 4, 2022

(54) Treatment facility, in particular for the treatment of organic waste by fermentation

(71) Applicant: Institut national de recherche pour l'agriculture, l'alimentation et l'environnement, PARIS (FR)

(72) Inventors: Laurent BLONDEL, SERVON SUR VILAINE (FR); Axelle DEGUEURCE, LA CHAPELLE CHAUSSEE (FR); Pascal PEU, RENNES (FR); Anne TREMIER, GOSNE (FR)

(21) Appl. No.: 17/617,575

(22) PCT Filed: Jun. 5, 2020
(86) PCT No.: PCT/FR2020/050964

§ 371 Date: Dec. 8, 2021

(30) Foreign Application Priority Data
Jun. 17, 2019 (FR) FR1906442

(51) Int. Cl. C12M 1/107; C12M 1/12; C12M 3/00; C12M 1/26

(52) U.S. Cl. CPC C12M 21/04; C12M 33/14; C12M 23/48; C12M 23/06

(57) ABSTRACT

The invention relates to a facility (1) for treating solid organic waste comprising a tubular reactor (2) equipped with an inlet (3) for supplying products to be treated, an outlet (4) for discharging treated products, and a biogas discharge outlet (5), and a frame that supports the reactor (2). The reactor is configured to form an inclined reactor with a slope that descends from the supply inlet (3) toward the discharge outlet (4). The reactor (2) comprises a tubular body (6) and a liquid/solid separator (7) extending axially inside the body (6), the liquid/solid separator (7) comprises a pipe (8) defined by a perforated peripheral lateral wall (9), surrounded by the body (6) to house a space (10) for collecting liquid between the perforated peripheral lateral wall and the body, and the reactor (2) comprises an inlet orifice (11) that can communicate with the interior of the pipe (8) of the liquid/solid separator (7) and an outlet orifice (12) in fluid communication with the liquid collection space (10), and connected to the inlet orifice (11) by a forced fluid circulation circuit (13) arranged at least partially outside the body (6) of the reactor (2).

The present invention relates to a treatment facility, notably for the treatment by fermentation, in particular anaerobic fermentation, of solid products that are at least partially fermentable, in particular solid organic waste, notably in order to produce biogas.

Biogas means a combustible gas produced by fermenting organic matter in the absence of oxygen.

More specifically, the invention relates to a treatment facility including a tubular reactor, said tubular reactor being fitted with at least one untreated-product feed inlet, a first outlet referred to as the treated-product discharge outlet and a second outlet referred to as the biogas discharge outlet.

The transformation of organic matter into biogas, such as methane, by anaerobic fermentation of said organic matter is a biological process well known to the person skilled in the art. Such anaerobic fermentation, also referred to as methanization, is a proven technology used to derived value from liquid organic effluent, but is a less efficient means for deriving value from solid organic waste. Notably, one of the problems of treating solid organic waste relates to the difficulty of bringing microorganisms into contact with nutrients in an environment where mechanical agitation is difficult. Methanization facilities based on recycling solid organic waste are nonetheless becoming increasingly common. In particular, there is a pressing need for local methanization facilities. Such facilities for treating solid organic waste by anaerobic fermentation are referred to as local facilities to distinguish them from industrial treatment facilities. Unlike industrial facilities, such local treatment facilities are intended to treat a smaller quantity of waste, usually in the order of several hundred tons of waste per year. This imposes a number of conditions on such treatment facilities, specifically: small size, low maintenance, simplified use, continuous high-level production of biogas combined with low water and energy consumption. The facilities for treating solid organic waste by anaerobic fermentation currently available on the market do not fulfill the aforementioned conditions. This is the case for the facility described in U.S. Pat. No. 5,782,950. In this document, the facility has two reactors. The first reactor is an inclined reactor that receives solid waste and performs a liquid/solid separation by pressing said waste. Inside this reactor, solid waste is moved mechanically by a conveyor screw from the lower end to the upper end of the reactor and the liquid is outputted at the upper end of the reactor. No biogas is produced by anaerobic fermentation of the solid products in this first reactor. The facility therefore includes a second reactor, where the biogas is produced. This makes the facility significantly larger. U.S. Pat. No. 5,159,694 describes a rotary reactor facility that is incompatible with low energy cost.

One objective of the invention is to propose a treatment facility of the aforementioned type that is designed to ensure the near-continuous production of biogas combined with production of a reduced quantity of digestate without adversely affecting ease of use of the facility or low water consumption.

For this purpose, the invention relates to a treatment facility, notably for the treatment by fermentation of solid products that are at least partially fermentable, in particular solid organic waste, notably in order to produce biogas, said facility including a tubular reactor and a support chassis for said tubular reactor, said tubular reactor being provided with at least one untreated-product feed inlet, a first outlet referred to as the treated-product discharge outlet, and a second outlet referred to as the biogas discharge outlet, characterized in that the tubular reactor has at least one configuration in which the untreated-product feed inlet of the reactor is positioned at a level above the treated-product discharge outlet of the reactor to form an inclined reactor sloping downwards from the untreated-product feed inlet towards the treated-product discharge outlet of the reactor, in which the tubular reactor comprises a tubular body and a liquid/solid separator extending axially inside said body, in which the liquid/solid separator includes a duct internally delimiting a receiving space for receiving the untreated products, this duct being a duct delimited by a perforated peripheral side wall, this perforated peripheral side wall being surrounded by said body to form a liquid collection space between said perforated peripheral side wall and the body, and in that the reactor also has an inlet orifice that can communicate with the inside of the duct of the liquid/solid separator and an outlet orifice in fluid communication with the liquid collection space, the inlet orifice being arranged above the outlet orifice in the configuration in which the reactor is inclined downwards from the untreated-product feed inlet towards the treated-product discharge outlet of the reactor, this inlet orifice being linked to the outlet orifice by a fluid flow circuit arranged at least partially outside the body of the reactor, this fluid flow circuit being fitted with at least one system, such as a pump, to force fluid flow from the outlet orifice to the inlet orifice. The tubular reactor is designed such that, in at least one configuration of the tubular reactor, the tubular reactor is an inclined reactor that helps to limit the size of the facility while enabling gravity flow inside said reactor. The presence of an inclined tubular reactor containing a liquid/solid separator combined with a forced flow circuit for a fluid, in particular a liquid, from the outlet orifice to the inlet orifice of the tubular reactor enables, without human intervention and concurrently with the production of biogas, the liquid to be separated from the solid and the separated liquid to be recirculated inside the reactor, thereby providing a regular supply of liquid inoculum or leachate inside the reactor, promoting the fermentation of the solid products. The separation and recirculation of liquids helps to improve the efficiency of the degradation of the solid products and to reduce the quantity of leftover solid matter, referred to as digestate, that is produced. This design also obviates the need for an additional separation phase for the digestate.

According to one embodiment of the invention, the reactor is mounted on the support chassis with adjustable inclination to vary the difference in level between the untreated-product feed inlet and the treated-product discharge outlet of said reactor. The ability to adjust the inclination of the tubular reactor facilitates the loading of said untreated products into said reactor by simply tilting the reactor to lower the untreated-product feed inlet, making said inlet more easily accessible.

According to one embodiment of the invention, the reactor is mounted on the support chassis with adjustable inclination by means of a pivot link between the support chassis and the reactor, this pivot link having a horizontal pivot axis extending transversely to the longitudinal axis of the tubular body of the reactor and horizontally when said facility is positioned on a flat horizontal surface.

According to one embodiment of the invention, said facility includes a drive system for pivoting the reactor in relation to the support chassis. This drive system can be motorized or actuated manually.

According to one embodiment of the invention, said facility includes an untreated-product feed system of the reactor.

According to one embodiment of the invention, the untreated-product feed system includes one or more fenestrated storage cassettes for untreated products that can be positioned in the duct of the liquid/solid separator and moved slidingly in the duct of the liquid/solid separator.

According to one embodiment of the invention in which the facility has several cassettes, the cassettes are designed to be arranged in line with one another inside the duct of the liquid/solid separator. This arrangement enables a cassette to be discharged from one end of the duct of the liquid/solid separator as a cassette is inserted via the opposite end of said duct.

According to one embodiment of the invention, the product feed system includes a pusher (19) mounted slidingly inside the reactor. Thus, where the feed system has one or more cassettes, and when the or at least one of the cassettes is positioned in the duct of the liquid/solid separator, said cassette can be moved slidingly in said duct of the liquid/solid separator by a pushing action exerted by said pusher on said cassette. In a variant, the pusher can act by pushing the untreated products arranged loose inside the duct of the liquid/solid separator.

According to one embodiment of the invention, the pusher has a piston head with an active pushing surface, this active pushing surface of the piston head being provided with a hollow rod projecting from the active surface of the piston head, this rod having perforations over at least one portion of the length thereof that are in fluid communication with the inlet orifice of the fluid flow circuit in at least one of the possible positions of the pusher inside the reactor to form a spray rod. This design of the pusher enables fluid to be supplied inside the reactor and over a large portion of the length of the reactor.

According to one embodiment of the invention, the piston head of the pusher is fitted, on the side of the piston head opposite the side with the active pushing surface of said piston head, with a movement member preferably projecting at least partially from the untreated-product feed inlet of the reactor to enable the pusher to slide inside the reactor.

According to one embodiment of the invention, when the pusher is in the end-of-travel position inside the reactor, the piston head is arranged between the untreated-product feed inlet and the treated-product discharge outlet of the reactor and the pusher can be removed from the reactor via the untreated-product feed inlet of the reactor.

According to one embodiment of the invention, the fluid flow circuit linking the outlet orifice and the inlet orifice of the reactor is provided with a storage tank. This tank can be used as a storage tank for a volume of liquid referred to as the inoculum at the beginning of the fermentation process, then as a storage tank for leachate during the fermentation process. Liquefaction of the solid matter during the fermentation process enables the continuous production of leachate, which is added to the volume of liquid initially provided. This storage tank can be provided with a drainage member to maintain an optimum fill level of said tank.

According to one embodiment of the invention, the untreated-product feed inlet is an axial inlet and the untreated-product feed inlet and the treated-product discharge outlet of the reactor are each provided with an airtight closure system, and the body of the reactor and the duct of the liquid/solid separator are coaxial.

According to one embodiment of the invention, the body of the reactor is delimited by a double wall and is fitted with heating means.

### SHORT DESCRIPTION OF THE DRAWINGS

The invention is detailed in the description of example embodiments provided below with reference to the attached drawings, in which:

FIG. 1 is a cross-section view of a facility according to the invention,

FIG. 2 shows a facility according to the invention,

FIG. 3 is an exploded cross-section view of the elements forming a cassette,

FIG. 4 is a side view of the cassette in FIG. 3, and

FIG. 5 is a partial cross-section view of the reactor at the inlet orifice of the reactor.

As mentioned above, the treatment facility 1 according to the invention is more specifically designed to treat solid organic waste by anaerobic fermentation, for example urban waste, in particular kitchen waste from homes, restaurants and markets, including preparation waste (peel, pulp) through to meal waste, preferably at a rate of 50 to 200 tons per year.

This facility in particular enables the production of combustible gases comprising essentially methane or carbon dioxide, known as biogases, from the fermentation of organic matter in the absence of oxygen.

This facility 1 includes a tubular reactor 2 and a support chassis 15 for said tubular reactor 2. This tubular reactor 2 comprises a tubular body 6 and an untreated-product feed inlet 3, the tubular body 6 being an elongate body having any cross section. In the examples shown, this tubular body 6 is a cylindrical body having a circular section.

The untreated-product feed inlet 3 is an axial inlet formed at one end of the tubular body 6.

This reactor 2 also includes a first outlet 4 referred to as the treated-product discharge outlet 4, and a second outlet 5 referred to as the biogas discharge outlet 5.

In the examples shown, the treated-product discharge outlet 4 is an axial outlet arranged at the end of the tubular body 6 opposite the end provided with the untreated-product feed inlet 3, and the biogas discharge outlet 5 is a lateral outlet.

The untreated-product feed inlet 3 and the treated-product discharge outlet 4 of the reactor 2 are each fitted with an airtight closing system 27.

In the examples shown, each closing system 27 is a pneumatic pinch valve that can be inflated to switch from the open position to the closed position.

The body 6 of the tubular reactor 2 is, over at least one portion of the length thereof, delimited by a double wall 28 and provided with heating means 29.

In the examples shown, these heating means 29 comprise a hot-water circuit, of which at least one portion is formed by the free space between the two walls of the double wall of the body 6 of the tubular reactor.

This heating circuit is fed with hot fluid from a hot-fluid tank positioned on the support chassis 15. This hot-fluid tank is provided with members for heating said fluid, such as electrical resistors.

This tubular reactor 2 has at least one configuration in which the untreated-product feed inlet 3 of the reactor is positioned at a level above the treated-product discharge outlet 4 of the reactor 2 to form a reactor 2 inclined downwards from the untreated-product feed inlet 3 towards the treated-product discharge outlet 4 of the reactor 2.

In particular, in the examples shown, the reactor 2 is mounted on the support chassis 15 with adjustable inclination, by means of a pivot link 16 between the support chassis 15 and the reactor 2, to vary the difference in level between the untreated-product feed inlet 3 and the treated-product discharge outlet 4 of said reactor 2, this pivot link 16 having a horizontal pivot axis extending transversely to the longitudinal axis of the tubular body 6 of the reactor and horizontally when said facility is positioned on a flat horizontal surface.

The option of tilting the tubular reactor 2 by pivoting about a horizontal pivot axis provides a large number of advantages. This helps to reduce the size of the reactor, enables gravity movement inside said reactor, and facilitates the loading of untreated products into the reactor.

To facilitate this pivoting movement of the reactor 2 in relation to the support chassis 15, the facility can include a manually actuated or automatic drive system 17 for pivoting the reactor 2 in relation to the support chassis 15.

In the examples shown, the pivot through which the pivot axis of the pivot link 16 between the support chassis 15 and the reactor 2 passes is constrained to rotate with a notched wheel. This notched wheel engages with a drive sprocket via a handle and is carried by the support chassis 15. The gear wheel, the sprocket and the handle form the drive system 17.

The tubular reactor 2 also includes a liquid/solid separator 7 extending axially inside the body 6. This liquid/solid separator 7 includes a duct 8 internally delimiting a receiving space for receiving the untreated products. This duct 8, which is an elongated body open at each of the ends thereof, is a perforated duct delimited by a perforated peripheral side wall 9. In the examples shown, this duct 8 is a cylindrical duct having a circular section.

Preferably, the perforations of the duct 8 that are closest to the product feed inlet 3 of the reactor 2 are larger than the perforations that are closest to the treated-product discharge outlet 4.

Preferably, the size of the perforations lessens gradually from the product feed inlet 3 to the treated-product discharge outlet 4.

Preferably, the body 6 of the reactor 2 and the duct 8 of the separator 7 are coaxial, as in the example shown.

The perforated side wall 9 delimiting the duct 8 of the liquid/solid separator 7 is surrounded by the body 6 of the reactor 2 to form a liquid collection space 10 between the perforated peripheral side wall 9 and the body 6 of the reactor 2.

The reactor 2 also includes an inlet orifice 11 that can communicate with the inside of the duct 8 of the liquid/solid separator 7 and an outlet orifice 12 in fluid communication with the liquid collection space 10.

The inlet orifice 11 is arranged above the outlet orifice 12 in the configuration in which the reactor is inclined downwards from the untreated-product feed inlet 3 towards the treated-product discharge outlet 4 of the reactor 2.

This inlet orifice 11 is linked to the outlet orifice 12 by a fluid flow circuit 13 arranged at least partially, and in this case entirely, outside the body 6 of the reactor 2.

This fluid flow circuit 13 is fitted with a system 14, such as a pump, to force fluid flow from the outlet orifice 12 to the inlet orifice 11 of the reactor.

This fluid flow circuit 13 is therefore a recirculation circuit that enables recirculation of the liquid that has percolated through the untreated solid products, also referred to as leachate, and the recirculation of the liquid resulting from the liquefaction of at least a portion of the untreated solid products, such liquefaction resulting from fermentation.

This fluid flow circuit 13 is preferably provided with a filter 30 at the outlet orifice 12 of the reactor 2 that is used to catch undegraded particles liable to block the fluid flow circuit 13.

This fluid flow circuit 13 linking the outlet orifice 12 and the inlet orifice 11 of the reactor is provided with a storage tank 26.

This liquid storage tank 26 can be pre-filled with inoculum when the facility is started up. This inoculant can be any liquid digestate taken from a nearby methanization unit. Preferably, the digestate used as inoculum is taken from a site primarily treating kitchen biowaste.

The leachate coming from the reactor 2 then passes through this storage tank 26 before being reinjected into the reactor 2.

This storage tank 26 can be provided with a drainage or overflow system to control the fill level of said tank. The leachate extracted from this storage tank 26 can be reused.

To facilitate the tilting of the reactor 2, a portion of the circuit can be a concertina duct or a flexible duct.

Although the facility 1 can be fed with loose untreated products, this solution is not preferred. Consequently and preferably, the facility 1 includes an untreated-product feed system 18 of the reactor 2.

This feed system 18 can be provided in a wide variety of forms. In particular, the feed system 18 can include one or more fenestrated storage cassettes 20 for untreated products that can be positioned in the duct 8 of the liquid/solid separator 7 and moved slidingly in the duct 8 of the liquid/solid separator 7.

In the examples shown, the solution of a treatment facility 1 with several cassettes 20 has been chosen.

The cassettes 20 are designed to be arranged in line with one another inside the duct 8 of the liquid/solid separator 7. The cassettes then form a line of cassettes such that all of the cassettes in the line move if one end of the line of cassettes is pushed.

This arrangement enables a cassette to be discharged from one end of the liquid/solid separator as a cassette is inserted via the opposite end of the liquid/solid separator.

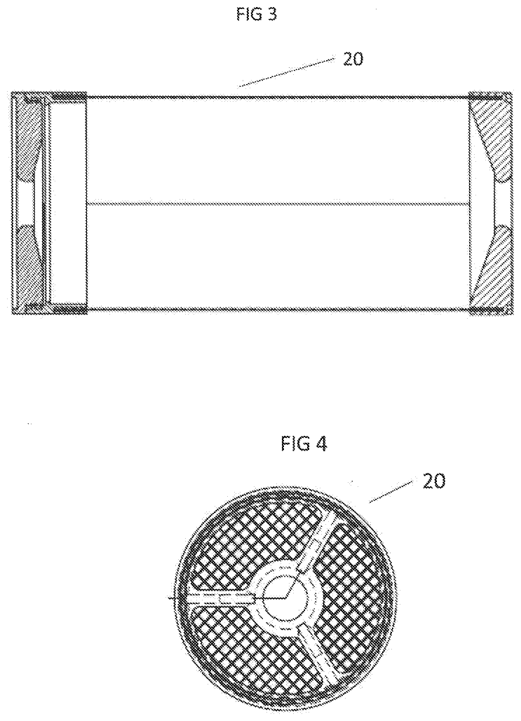

As shown in FIG. 3, each cassette 20 is a tubular body, in particular a cylindrical body, in which the wall defining the body is perforated, and at least one cover closing one end of said body, this cover being fenestrated.

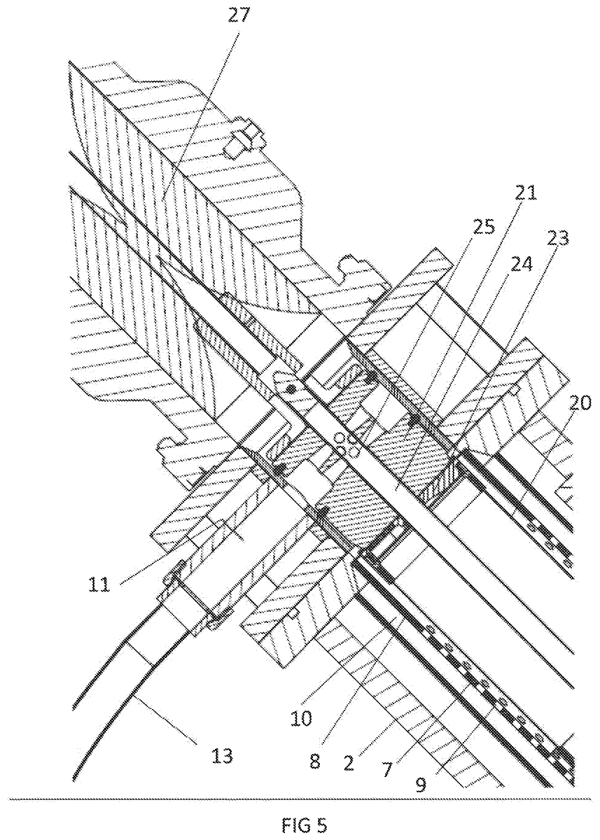

In the examples shown, this cover is formed by a grille, as shown in FIG. 4. The untreated products are inserted into the body of the cassette once the cover has been opened. The cassette 20 can be inserted into the reactor 2 once the cover has been closed.

To facilitate the movement of the untreated products, and in particular the movement of each cassette inside the reactor 2, the product feed system 18 includes a pusher 19 mounted slidingly inside the reactor 2. This pusher 19 enables each cassette, when positioned in the duct of the liquid/solid separator, to be moved slidingly in said duct of the liquid/solid separator under the action of a push exerted by said pusher on said cassette. Such a pusher can also be provided and where the reactor is fed in bulk.

In the examples shown, this pusher 19 is a removable pusher that can be removed from or inserted into the reactor 2 via the untreated-product feed inlet 3 of the reactor 2.

This pusher 19 includes a piston head 21. This piston head 21 has an active pushing surface 23, corresponding to the surface of the piston head 21 that can be brought to bear against a cassette 20 to be moved, in this case a cassette at the end of the previously formed line of cassettes.

This piston head 21 of the pusher is provided, on the side of the piston head 21 opposite the side with the active pushing surface of said piston head, with a movement member 22 projecting at least partially from the untreated-product feed inlet 3 of the reactor 2 to enable the pusher 19 to be moved slidingly inside the reactor 2, driven in axial movement by the movement member 22 of the pusher 19, which is in this case an elongate body.

This piston head 21 sliding inside the body 6 of the reactor 2 is in the end-of-travel position of the pusher 19 inside the reactor 2, between the untreated-product feed inlet 3 and the treated-product discharge outlet 4 of the reactor 2.

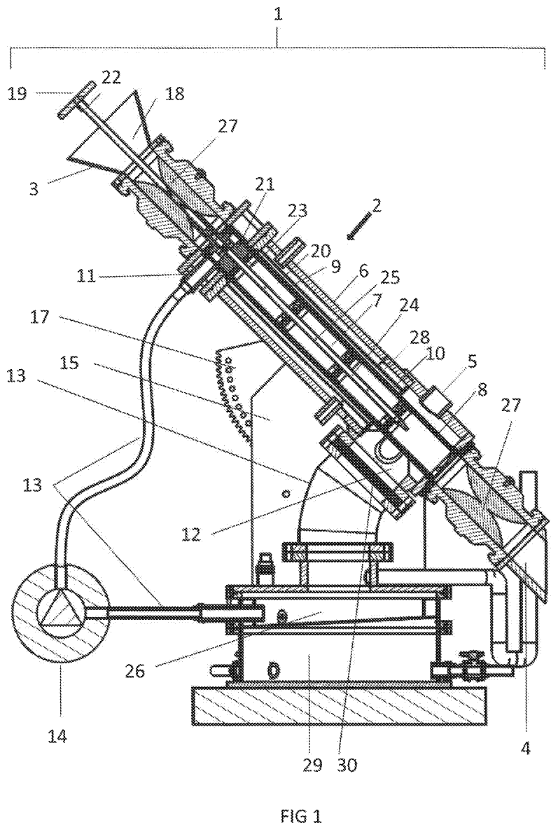

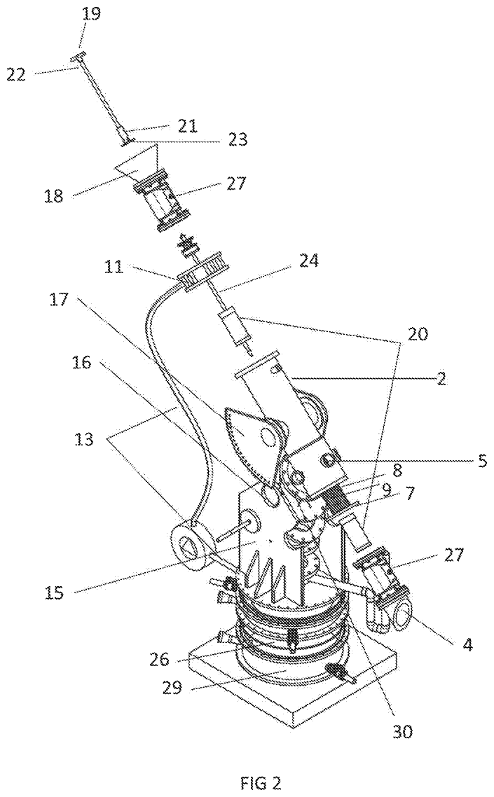

In particular, this piston head 21 is in the end-of-travel position at the inlet orifice 11 of the tubular reactor 2 to enable fluid communication between the fluid flow circuit 13 and the inside of the separator duct 8. For this purpose, the piston head 21 can be provided internally with at least one duct bringing the fluid flow circuit 13 into communication with the inside of the duct 8 of the separator 7, as shown in FIG. 1.

In the examples shown, the active pushing surface 23 of the piston head 21 is provided with a hollow rod 24 projecting axially from the active surface 23 of the piston head 21.

This rod 24 has perforations 25 over at least one portion of the length thereof and is in fluid communication with the inlet orifice 11 of the fluid flow circuit 13 in at least one of the possible positions of the pusher 19 inside the reactor 2, to form a spray rod. This spray rod also acts as a pin on which the or at least one of the cassettes 20 can be positioned, if cassette-feed is used. In summary, in the example shown, the pusher 19 has a piston head 21 with an active pushing surface 23 corresponding to the surface of said piston head 21 that can be brought to bear against a cassette 20 to be moved, this active pushing surface 23 of the piston head 21 being provided with a hollow rod 24 projecting from the active surface 23 of the piston head 21, this rod 24 having perforations 25 over at least one portion of the length thereof that are in fluid communication with the inlet orifice 11 of the fluid flow circuit 13 in at least one of the possible positions of the pusher 19 inside the reactor 2, to form a spray rod, this spray rod also acting as a pin on which the or at least one of the cassettes 20 can be positioned.

Thus, each cassette 9 is provided with an axial through-hole to enable the cassette to be positioned on the spray rod acting as a pin. This spray rod provides numerous advantages and in particular enables the inoculum or leachate to be distributed over a significant length of the reactor 2.

This spray rod 24 is in fluid communication with the inlet orifice 11 of the fluid flow circuit 13 when the pusher 19 is in the end-of-travel position inside the reactor 2.

Such a facility works as follows: the cassettes are pre-filled with products, in particular solid urban waste such as kitchen waste to be treated. The storage tank 26 is filled with an inoculum.

The pusher 19 is removed from the reactor 2.

The cassettes are positioned on the spray rod of the pusher.

The reactor is pivoted to lower the untreated-product feed inlet 3.

The pusher carrying the cassettes is inserted via the untreated-product feed inlet 3 into the reactor 2.

The cassettes are inserted using the pusher, sliding inside the reactor in the liquid/solid separator 7. At the end-of-travel position, the inlet orifice 11 of the reactor 2 is in fluid communication with the duct 8 of the liquid/solid separator 7 such as to feed fluid into the latter via the spray rod 24 passing axially through each cassette 20.

Once the pusher and the cassettes are inside the reactor 2, the product feed inlet 3 and the treated-product discharge outlet 4 are closed and sealed using closing systems 27 to gradually establish the anaerobic conditions required for fermentation in order to produce biogas.

The reactor is pivoted to raise the untreated-product feed inlet 3.

The fluid flow pump 14 is started to enable the content of the storage tank 26 to be fed into the reactor 2 via the inlet orifice 11 of the reactor 2.

The heating means are activated to enable circulation of a hot fluid in the double wall of the reactor 2. Furthermore, the walls of the storage tank 26 that is positioned on the fluid flow circuit 13 can be heated.

The fluid coming from the flow circuit 13 enters the reactor 2 via the inlet orifice 11, feeds each cassette via the spray rod 24 fitted to the pusher 19, and percolates under the action of gravity through the untreated solid products. The fluid then passes through the solid products contained in the cassettes, and then passes through the perforated wall of the separator to reach the space 10 formed between the perforated peripheral side wall 9 of the separator 7 and the body 6.

When percolating through the untreated solid products, the liquid (specifically the inoculum or the leachate) collects solubilized organic matter, i.e. degraded and liquefied solid organic matter, before leaving the reactor 2 through the outlet orifice 12 and returning, via the filter 30 fitted to said outlet orifice 12 and the flow circuit 13, to the reactor 2, to be percolated again. When flowing through the fluid circuit 13, the liquid passes through the storage tank 26 arranged on said circuit 13.

Liquefaction of the solid matter enables the continuous production of leachate, which is added to the volume of inoculum initially provided.

Simultaneously, the degraded matter enables the production of biogas and left-over solid matter referred to as digestate.

When a new cassette needs to be inserted in the facility, the reactor is tilted to lower the untreated-product feed inlet 3 of the reactor, and the pump is stopped.

The untreated-product feed inlet 3 and the treated-product discharge outlet 4 of the reactor 2 are opened and the pusher is removed from the reactor 2 via the untreated-product feed inlet 3 of the reactor 2.

A new cassette filled with untreated products is positioned on the spray rod of the pusher as close as possible to the piston head of the pusher, and the pusher, carrying the related cassette, is reinserted into the reactor, such that the spray rod of the pusher passes through the cassettes already in place as the pusher is inserted slidingly into the reactor.

This pushing action exerted by the sliding movement of the pusher inside the reactor causes the last cassette in the line of cassettes to be ejected, i.e. the cassette positioned on the side of the treated-product discharge outlet 4 of the reactor 2.

The cycle as described above can then be repeated.

Thus, each time a new cassette of untreated products is inserted, a cassette of treated products is removed from the tubular reactor.

The insertion frequency of the cassettes determines the residence time of the products in the reactor. This treatment time is usually at least four weeks.

To maintain an optimal level of leachate in the storage tank 26, a portion thereof is regularly removed from the facility and can be used as liquid fertilizer.

A gauge or equivalent is used to monitor the level of leachate inside the storage tank 26.

This design enables the facility to produce biogas continuously since production is only interrupted sporadically for the time required to insert a cassette of untreated products and to remove a cassette of treated products.

This operation can take just a few minutes.

1. A treatment facility, notably for the treatment by fermentation of solid products that are at least partially fermentable, in particular solid organic waste, notably in order to produce biogas, said facility comprising:
a tubular reactor and a support chassis for said tubular reactor, said tubular reactor being provided with at least one untreated-product feed inlet, a first outlet referred to as the treated-product discharge outlet, and a second outlet referred to as the biogas discharge outlet,
wherein the tubular reactor has at least one configuration in which the untreated-product feed inlet of the reactor is positioned at a level above the treated-product discharge outlet of the reactor to form a reactor inclined downwards from the untreated-product feed inlet towards the treated-product discharge outlet of the reactor, in which the tubular reactor comprises a tubular body and a liquid/solid separator extending axially inside said body, in that the liquid/solid separator includes a duct internally delimiting a receiving space for receiving the untreated products, this duct being a duct delimited by a perforated peripheral side wall, this perforated peripheral side wall being surrounded by said body to form a liquid collection space between said perforated peripheral side wall and the body, and in that the reactor also has an inlet orifice that can communicate with the inside of the duct of the liquid/solid separator and an outlet orifice in fluid communication with the liquid collection space, the inlet orifice being arranged above the outlet orifice in the configuration in which the reactor is inclined downwards from the untreated-product feed inlet towards the treated-product discharge outlet of the reactor, this inlet orifice being linked to the outlet orifice by a fluid flow circuit arranged at least partially outside the body of the reactor, this fluid flow circuit being fitted with at least one system, such as a pump, to force fluid flow from the outlet orifice towards the inlet orifice.

2. The treatment facility, notably for the treatment by fermentation of solid products that are at least partially fermentable as claimed in claim 1, wherein the reactor is mounted on the support chassis with adjustable inclination to vary the difference in level between the untreated-product feed inlet and the treated-product discharge outlet of said reactor.

3. The treatment facility, notably for the treatment by fermentation of solid products that are at least partially fermentable as claimed in claim 2, wherein the reactor is mounted on the support chassis with adjustable inclination by means of a pivot link between the support chassis and the reactor, this pivot link having a horizontal pivot axis extending transversely to the longitudinal axis of the tubular body of the reactor and horizontally when said facility is positioned on a flat horizontal surface.

4. The treatment facility, notably for the treatment by fermentation of solid products that are at least partially fermentable as claimed in claim 3, wherein said facility includes a drive system for pivoting the reactor in relation to the support chassis.

5. The treatment facility, notably for the treatment by fermentation of solid products that are at least partially fermentable as claimed in claim 1, wherein said facility includes an untreated-product feed system of the reactor.

6. The treatment facility, notably for the treatment by fermentation of solid products that are at least partially fermentable as claimed in claim 5, wherein the untreated-product feed system includes one or more fenestrated storage cassettes for untreated products that can be positioned in the duct of the liquid/solid separator and moved slidingly in the duct of the liquid/solid separator.

7. The treatment facility, notably for the treatment by fermentation of solid products that are at least partially fermentable as claimed in claim 6, wherein, said facility having several cassettes, the cassettes are designed to be arranged in line with one another inside the duct of the liquid/solid separator.

8. The treatment facility, notably for the treatment by fermentation of solid products that are at least partially fermentable as claimed in claim 5, wherein the product feed system includes a pusher mounted slidingly inside the reactor.

9. The treatment facility, notably for the treatment by fermentation of solid products that are at least partially fermentable as claimed in claim 8, wherein the pusher has a piston head with an active pushing surface, this active pushing surface of the piston head being provided with a hollow rod projecting from the active surface of the piston head, this rod having perforations over at least one portion of the length thereof that are in fluid communication with the inlet orifice of the fluid flow circuit in at least one of the possible positions of the pusher inside the reactor to form a spray rod.

10. The treatment facility, notably for the treatment by fermentation of solid products that are at least partially fermentable as claimed in claim 9, wherein the piston head of the pusher is fitted, on the side of the piston head opposite the side with the active pushing surface of said piston head, with a movement member projecting at least partially from the untreated-product feed inlet of the reactor to enable the pusher to slide inside the reactor.

11. The treatment facility, notably for the treatment by fermentation of solid products that are at least partially fermentable as claimed in claim 9, wherein, when the pusher is in the end-of-travel position inside the reactor, the piston head is arranged between the untreated-product feed inlet and the treated-product discharge outlet of the reactor and in that the pusher can be removed from the reactor via the untreated-product feed inlet (3) of the reactor.

12. The treatment facility, notably for the treatment by fermentation of solid products that are at least partially fermentable as claimed in claim 1, wherein the fluid flow circuit linking the outlet orifice and the inlet orifice of the reactor is provided with a storage tank.

13. The treatment facility, notably for the treatment by fermentation of solid products that are at least partially fermentable as claimed in claim 1, wherein the untreated-product feed inlet is an axial inlet and in that the untreated-product feed inlet and the treated-product discharge outlet of the reactor are each provided with an airtight closing system, and in that the body of the reactor and the duct of the liquid/solid separator are coaxial.

14. The treatment facility, notably for the treatment by fermentation of solid products that are at least partially fermentable as claimed in claim 1, wherein the body of the reactor is delimited by a double wall and is fitted with heating means.
